(19) United States
(12) Patent Application Publication
(10) Pub. No.: US 2006/0172942 A1
(43) Pub. Date: Aug. 3, 2006

(54) Process for producing polypeptide mixtures using hydrogenolysis

(75) Inventor: Ben-Zion Dolitzky, Petach-Tikva (IL)

Correspondence Address: COOPER & DUNHAM, LLP, 1185 AVENUE OF THE AMERICAS, NEW YORK, NY, 10036, US

(73) Assignee: Teva Pharmaceutical Industries, Ltd.

(21) Appl. No.: 11/336,251

(22) Filed: Jan. 20, 2006

Related U.S. Application Data
(60) Provisional application No. 60/649,442, filed on Feb. 2, 2005.

(51) Int. Cl. A61K 38/17; C07K 1/02; C07K 14/00

(52) U.S. Cl. 514/12; 530/333; 530/324; 530/300

(57) ABSTRACT

The subject invention provides for a process for making a mixture of acetate salts of polypeptides, each of which consisting of glutamic acid, alanine, tyrosine and lysine, wherein the mixture has a desired peak molecular weight, comprising: a) polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight; b) removing the benzyl protecting group from the mixture of protected polypeptides by contacting the polypeptides with a hydrogenolysis catalyst and hydrogen to produce a mixture of trifluoroacetyl protected polypeptides, which mixture of polypeptides in unprotected form having the first peak molecular weight; c) removing the trifluoroacetyl protecting group from the trifluoroacetyl protected polypeptides by contacting the polypeptides with an organic base solution to form a mixture of polypeptides, which mixtures of polypeptides in unprotected form having the first peak molecular weight; d) removing the free trifluoroacetyl groups and low molecular weight impurities by ultrafiltration to obtain the mixture of polypeptides each of which consisting of glutamic acid, alanine, tyrosine and lysine; and e) contacting the mixture of polypeptides each of which consisting of glutamic acid, alanine, tyrosine and lysine with an aqueous solution of acetic acid to form the mixture of acetate salts of polypeptides each of which consisting of glutamic acid, alanine, tyrosine and lysine and having the desired peak molecular weight.

This application claims the benefit of U.S. Provisional Application No. 60/649,442, filed Feb. 2, 2005, the entire contents of which are hereby incorporated by reference.

Throughout this application various publications are referenced by their full citations. The disclosures of these publications in their entireties are hereby incorporated by reference into this application in order to more fully describe the state of the art to which this invention pertains.

### BACKGROUND OF THE INVENTION

Glatiramer acetate (GA) is a mixture of polypeptides which has been approved for the treatment of multiple sclerosis. COPAXONE®, the brand name for a pharmaceutical composition which contains glatiramer acetate (GA) as the active ingredient, contains the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively. The average molecular weight of glatiramer acetate is 4,700-11,000 daltons. Chemically, glatiramer acetate is designated L-glutamic acid polymer with L-alanine, L-lysine and L-tyrosine, acetate (salt). Its structural formula is:
(Glu, Ala, Lys, Tyr)ₓ.χCH₃COOH (C₅H₉NO₄.C₃H₇NO₂.C₆H₁₄N₂O₂. C₉H₁₁NO₃)ₓ.χC₂H₄O₂CAS-147245-92-9

(“Copaxone”, Physician's Desk Reference, (2000), Medical Economics Co., Inc., (Montvale, N.J.), 3115.)

Processes of manufacturing polypeptides of this type, including glatiramer acetate, are described in U.S. Pat. No. 3,849,550, issued Nov. 19, 1974 to Teitelbaum, et al., U.S. Pat. No. 5,800,808, issued Sep. 1, 1998 to Konfino, et al., and PCT International Publication No. WO 00/05250, published Feb. 3, 2000 (Aharoni, et al.) which are hereby incorporated by reference. For example, polypeptides of this type were prepared from the N-carboxyanhydrides of tyrosine, alanine, γ-benzyl glutamate and ε-N-trifluoro-acetyllysine. The polymerization was carried out at ambient temperature in anhydrous dioxane with diethylamine as initiator. The deblocking of the γ-carboxyl group of the glutamic acid was affected by hydrogen bromide (HBr) in glacial acetic acid and is followed by the removal of the trifluoroacetyl groups from the lysine residues by 1 M piperidine (U.S. Pat. No. 3,849,550, issued Nov. 19, 1974 to Teitelbaum, et al.).

The deprotection of the γ-carboxyl group of the glutamic acid requires the use of large amounts of HBr/acetic acid. As a result, a large volume of acidic waste is produced. The disposal of this acidic waste is difficult and costly. Alternate methods of production of such polypeptides are desirable in order to eliminate the problems of acidic waste products.

### SUMMARY OF THE INVENTION

The subject invention provides for a process for making a mixture of acetate salts of polypeptides, each of which consisting of glutamic acid, alanine, tyrosine and lysine, wherein the mixture has a desired peak molecular weight, comprising:
a) polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight;
b) removing the benzyl protecting group from the mixture of protected polypeptides by contacting the polypeptides with a hydrogenolysis catalyst and hydrogen to produce a mixture of trifluoroacetyl protected polypeptides, which mixture of polypeptides in unprotected form having the first peak molecular weight;
c) removing the trifluoroacetyl protecting group from the trifluoroacetyl protected polypeptides by contacting the polypeptides with an organic base solution to form a mixture of polypeptides, which mixtures of polypeptides in unprotected form having the first peak molecular weight;
d) removing the free trifluoroacetyl groups and low molecular weight impurities by ultrafiltration to obtain the mixture of polypeptides each of which consisting of glutamic acid, alanine, tyrosine and lysine; and
e) contacting the mixture of polypeptides each of which consisting of glutamic acid, alanine, tyrosine and lysine with an aqueous solution of acetic acid to form the mixture of acetate salts of polypeptides each of which consisting of glutamic acid, alanine, tyrosine and lysine and having the desired peak molecular weight.

The subject invention also provides for a process for making a mixture of trifluoroacetyl protected polypeptides, each of which consisting of glutamic acid, alanine, tyrosine and trifluoroacetyllysine, wherein the mixture of polypeptides in unprotected form has a first peak molecular weight, comprising:
a. polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight; and
b. removing the benzyl protecting group from the mixture of protected polypeptides by contacting the polypeptides with a hydrogenolysis catalyst and hydrogen, to obtain the mixture of trifluoroacetyl protected polypeptides each of which consisting of glutamic acid, alanine, tyrosine and trifluoroacetyllysine and which mixture of polypeptides in unprotected form having the first peak molecular weight.

### DETAILED DESCRIPTION OF THE INVENTION

The subject invention provides for a process for making a mixture of acetate salts of polypeptides, each of which consisting of glutamic acid, alanine, tyrosine and lysine, wherein the mixture has a desired peak molecular weight, comprising:
a) polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight;
b) removing the benzyl protecting group from the mixture of protected polypeptides by contacting the polypeptides with a hydrogenolysis catalyst and hydrogen to produce a mixture of trifluoroacetyl protected polypeptides, which mixture of polypeptides in unprotected form having the first peak molecular weight;
c) removing the trifluoroacetyl protecting group from the trifluoroacetyl protected polypeptides by contacting the polypeptides with an organic base solution to form a mixture of polypeptides, which mixtures of polypeptides in unprotected form having the first peak molecular weight;
d) removing the free trifluoroacetyl groups and low molecular weight impurities by ultrafiltration to obtain the mixture of polypeptides each of which consisting of glutamic acid, alanine, tyrosine and lysine; and
e) contacting the mixture of polypeptides each of which consisting of glutamic acid, alanine, tyrosine and lysine with an aqueous solution of acetic acid to form the mixture of acetate salts of polypeptides each of which consisting of glutamic acid, alanine, tyrosine and lysine and having the desired peak molecular weight.

In an embodiment, the first peak molecular weight may be 2,000 daltons to 40,000 daltons, or 2,000 daltons to 20,000 daltons or 4,000 daltons to 8,600 daltons or 4,000 daltons to 8,000 daltons or 6,250 daltons to 8,400 daltons or 2,000 daltons to 13,000 daltons or 4,700 daltons to 13,000 daltons or 10,000 daltons to 25,000 daltons or 15,000 daltons to 25,000 daltons or 18,000 daltons to 25,000 daltons or 20,000 daltons to 25,000 daltons or 4,700 daltons to 11,000 daltons or 7,000 daltons or 13,000 daltons to 18,000 daltons or 15,000 daltons or 12,500 daltons.

In an embodiment, the desired peak molecular weight may be 2,000 daltons to 40,000 daltons or 2,000 daltons to 20,000 daltons or 4,000 daltons to 8,600 daltons or 4,000 daltons to 8,000 daltons or 6,250 daltons to 8,400 daltons or 2,000 daltons to 13,000 daltons or 4,700 daltons to 13,000 daltons or 10,000 daltons to 25,000 daltons or 15,000 daltons to 25,000 daltons or 18,000 daltons to 25,000 daltons or 20,000 daltons to 25,000 daltons or 4,700 daltons to 11,000 daltons or 7,000 daltons or 13,000 daltons to 18,000 daltons or 15,000 daltons or 12,500 daltons.

In an embodiment, the hydrogenolysis catalyst may be Palladium/carbon, Raney Nickel, Pt, Pt/C, PtO₂, Pd(OH)₂, Rh/C, or RhCl(PPh₃)₃.

In another embodiment, the hydrogenolysis catalyst may be Palladium/carbon.

In yet another embodiment, the weight ratio of protected polypeptide to palladium/carbon catalyst may be 10:1.

In an embodiment, the step of contacting the polypeptides with the hydrogenolysis catalyst may be performed in a solvent selected from the group consisting of methanol, ethanol or isopropanol.

In another embodiment, the solvent may be methanol.

In an embodiment, the initiator may be a primary amine, a dialkyl amine or sodium methoxide.

In another embodiment, the initiator may be diethylamine.

In yet another embodiment, the amount of initiator may be 0.05% to 19% by weight or 0.1% to 17% by weight or 0.5% to 15% by weight or 1% to 10% by weight or 2% to 5% by weight or 2% by weight or 5% by weight.

In an embodiment, the organic base in step c) may be an aqueous organic base.

In another embodiment, the aqueous organic base may be a primary, secondary or tertiary amine or methanolic ammonia.

In yet another embodiment, the aqueous organic base may be piperidine.

The subject invention also provides for a mixture of acetate salts of polypeptides made by the previous processes.

The subject invention further provides for a pharmaceutical composition comprising the previous mixture and a pharmaceutically acceptable carrier.

The subject invention still further provides for a process for preparing a pharmaceutical composition comprising mixing the previous mixture with a pharmaceutically acceptable carrier.

The subject invention further provides for a process for preparing a pharmaceutical composition containing an aqueous mixture of acetate salts of polypeptides each of which consisting of glutamic acid, alanine, tyrosine and lysine, wherein the mixture has a desired peak molecular weight, the improvement comprising making the mixture of acetate salts of polypeptides by any one of the previous processes.

The subject invention provides for a process for making a mixture of trifluoroacetyl protected polypeptides, each of which consisting of glutamic acid, alanine, tyrosine and trifluoroacetyllysine, wherein the mixture of polypeptides in unprotected form has a first peak molecular weight, comprising:
a) polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight; and
b) removing the benzyl protecting group from the mixture of protected polypeptides by contacting the polypeptides with a hydrogenolysis catalyst and hydrogen, to obtain the mixture of trifluoroacetyl protected polypeptides each of which consisting of glutamic acid, alanine, tyrosine and trifluoroacetyllysine and which mixture of polypeptides in unprotected form having the first peak molecular weight.

In an embodiment, the hydrogenolysis catalyst may be Palladium/carbon, Raney Nickel, Pt, Pt/C, PtO₂, Pd(OH)₂, Rh/C, or RhCl(PPh₃)₃.

In another embodiment, the hydrogenolysis catalyst may be Palladium/carbon.

In yet another embodiment, the weight ratio of protected polypeptide to palladium/carbon catalyst may be 10:1.

In an embodiment, the step of contacting the polypeptides with a hydrogenolysis catalyst may be performed in a solvent selected from the group consisting of methanol, ethanol or isopropanol.

In another embodiment, the solvent may be methanol.

In yet another embodiment, the initiator may be a primary amine, a dialkyl amine or sodium methoxide.

In an embodiment, the initiator may be diethylamine.

In another embodiment, the amount of initiator may be 0.05% to 19% by weight or 0.1% to 17% by weight or 0.5% to 15% by weight or 1% to 10% by weight or 2% to 5% by weight or 2% by weight or 5% by weight.

In an embodiment, the first peak molecular weight may be 2,000 daltons to 40,000 daltons or 2,000 daltons to 20,000 daltons or 4,000 daltons to 8,600 daltons or 4,000 daltons to 8,000 daltons or 6,250 daltons to 8,400 daltons or 2,000 daltons to 13,000 daltons or 4700 to 13,000 daltons or 10,000 daltons to 25,000 daltons or 15,000 daltons to 25,000 daltons or 18,000 daltons to 25,000 daltons or 20,000 daltons to 25,000 daltons or 4,700 daltons to 11,000 daltons or 7,000 daltons or 13,000 daltons to 18,000 daltons or 15,000 daltons or 12,500 daltons.

The subject invention also provides for a mixture of trifluoroacetyl protected polypeptides each of which consisting of glutamic acid, alanine, tyrosine and trifluoroacetyllysine produced by any one of the immediately preceding processes.

The subject invention also provides for a process of making a mixture of acetate salts of polypeptides, each of which consisting of glutamic acid, alanine, tyrosine and lysine, wherein the mixture has a desired peak molecular weight, comprising:
a) treating the previous mixture with an organic base solution,
b) removing the free trifluoroacetyl groups and low molecular weight impurities by ultrafiltration to obtain a mixture of polypeptides each of which consisting of glutamic acid, alanine, tyrosine and lysine, and
c) contacting the mixture of polypeptides with an aqueous solution of acetic acid to form the mixture of acetate salts of polypeptides, each of which consisting of glutamic acid, alanine, tyrosine and lysine having the desired peak molecular weight.

In an embodiment of the previous process, the organic base may be an aqueous organic base.

In another embodiment of the previous process, the aqueous organic base may be a primary, secondary or tertiary amine or methanolic ammonia.

In yet another embodiment of the previous process, the aqueous organic base may be piperidine.

### EXPERIMENTAL DETAILS

### EXAMPLE 1

### Synthesis of Poly[5-benzyl-1-Glu, N6-TFA-L-Lys, L-Ala, L-Tyr]

7.43 g of L-tyrosine N-carboxyanhydride were added to 260 ml of dioxane and the mixture was heated to 60° C. for 20 minutes and was then filtered. 34.61 g of N6-trifluoroacetyl-L-Lysine N-carboxyanhydride were added to 630 ml of dioxane and the solution was stirred at 20-25° C. for 15 minutes and was then filtered. 21.25 g of L-alanine N-carboxyanhydride were added to 395 ml of dioxane and the solution was stirred at 20-25° C. for 15 minutes and was then filtered. 14.83 g of 5-benzyl L-glutamate N-carboxyanhydride were added to 260 ml of dioxane and the solution was stirred at 20-25° C. for 10 minutes and was then filtered.

The solutions were combined in a 2L Erlenmeyer flask equipped with a mechanical stirrer. The solutions were stirred together for 5 minutes. 3.9 g of diethylamine was then added to the reaction mixture. The mixture was stirred for 24 hours at 23-27° C.

The reaction mixture was then added to 5L deionized water. The solid reaction product was filtered, washed and dried at 60° C. under vacuum. 65.6g of solid white-off-white powder was produced.

### EXAMPLE 2

### Deprotection (Hydrogenolysis) of Poly[5-benzyl-L-Glu, N6-TFA-L-Lys, L-Ala, L-Tyr] to form Poly[L-Glu, N6-TFA-L-Lys, L-Ala, L-Tyr]

18 g of the solid product synthesized as described in Example 1 were suspended in 540 ml of methanol. 1.8 g of wet palladium on charcoal (10% Pd on charcoal type 87 L Powder, Johnson Matthey—Precious Metals Division) was added. Hydrogenolysis was achieved by bubbling H₂ at 2 Atm. for 7 hours through the mixture. The mixture was filtered. The reaction mixture was concentrated to 270 ml and was added to 600 ml of water. The mixture was stirred for one hour and the mixture was filtered and dried to yield 14 g of white-off-white powder.

### EXAMPLE 3

### Removal of the Trifluoroacetyl Group to form Poly[L-Glu, L-Lys, L-Ala, L-Tyr]

9 g of the product synthesized in Example 2 were added to 540 ml of water. 60 ml of piperidine were added to the mixture, and the mixture was stirred at room temperature for 24 hours. The mixture was filtered and a clear filtrate with a yellowish tint was attained. Ultrafiltration was performed using a 5 kilodalton membrane, to remove all of the low-molecular weight impurities. After 6 cycles of ultrafiltration, the solution was acidified with acetic acid until a pH of 4.0 was achieved. Water was added and the solution was ultrafiltrated until a pH of 5.5 was attained. The solution was concentrated and lyophilized for 60 hours. 4.7 g of a white, lyophilized cake of Poly[L-Glu, L-Lys, L-Ala, L-Tyr] was attained.

### EXAMPLE 4

### Molecular Weight Analysis

The molecular weight of the product of Example 3 was determined using a Superose 12 HR Gel Permeation HPLC column, equipped with an UV detector. Phosphate buffer, pH 1.5 was used as the mobile phase.

The total retention time of the column was determined using 200 μl of acetone diluted with 1 ml of water. The column was calibrated using TV molecular weight markers using Millennium calculations which were described in US Pat. No. 6,514,938, issued Feb. 4, 2003 (Gad, et al.) (see specifically Example 2) hereby incorporated by reference.

After calibration, a solution of 5 mg/ml of the product of Example 3 was prepared. The peak maximum retention time was measured, and the peak molecular weight was determined to be 12,700 daltons.

### EXAMPLE 5

### Hydrolysis and Determination of Amino Acid Content

A sample solution was prepared using 10 mg of the polypeptide from Example 3 added to an arginine internal control solution. The sample solution was hydrolyzed using concentrated HCl containing 1% (w/v) phenol, under a N₂ atmosphere at 110° C. for 24 hours. Amino acid control solutions, each containing one of glutamate, alanine, tyrosine, and lysine HCl were prepared and hydrolyzed. The sample solution and the controls were derivatized with ortho-phthaldialdehyde.

The samples and controls were analyzed using a Merck LiChrosorb RP18 7 μm column equipped with an UV detector. The mobile phase was phosphate buffer pH 2.5/acetonitirile gradient. The molar fractions of the amino acids in the polypeptide sample were determined based on peak area.

| Amino acid | Molar fraction |
|---|---|
| Glutamic Acid | 0.138 |
| Alanine | 0.42 |
| Tyrosine | 0.099 |
| Lysine | 0.343 |

### EXAMPLE 6

### Formation of Acetate Salt

The product of any one of Examples 1-3 is contacted with an aqueous solution of acetic acid to form the polypeptide acetate salt.

### DISCUSSION

The inventors of the disclosed invention found that hydrogenolysis is effective in removing the benzyl groups from glutamate residues of the protected polypeptides. Specifically, the inventors of the instant invention found that the use of hydrogenolysis using a palladium/carbon catalyst is effective in removing the benzyl groups from glutamate residues to form a trifluoroacetyl polypeptide, which is protected by the trifluoroacetyl groups on the lysine residues. Catalyst, for example palladium/carbon, can be recovered and reused thereby eliminating waste. The trifluoroacetyl groups were subsequently removed from the lysine residues by piperidine.

Other hydrogenolysis catalysts may also be used to remove the benzyl groups from the glutamate residues. Such known hydrogenolysis catalysts are Raney Nickel, Pt, Pt/C, PtO₂, Pd(OH)₂, Rh/C, RhCl(PPh₃)₃, and other transition metal catalysts. The hydrogenolysis reaction can be performed at a temperature between 20° C. and 100° C. and a pressure between 1 atm and 100 atm.

Using hydrogenolysis instead of HBr/acetic acid to remove the benzyl groups, however, posed a further complication. When HBr/acetic acid is used, it serves the dual function of both removing the benzyl groups from the glutamate residues and cleaving the polypeptide to achieve a desired average molecular weight of the mixture. Hydrogenolysis, however, does not cleave the polypeptide. Therefore, inventors of the disclosed process further modified the production process to achieve the desired peak molecular weight by using specific amounts of the initiator of the polymerization reaction.

Initiators that can be used are n-hexylamine and other primary amines, diethylamine and other other dialkyl amines, or sodium methoxide or any combination of initiators. U.S. Pat. No. 5,800,808, issued Sep. 1, 1998 (Konfino, et al.) discloses the use of 0.1-0.2% diethylamine as an initiator in a process conducted at room temperature for 24 hours that also uses HBr to achieve polypeptides with a molecular weight in the range of 5000-9000 daltons. In contrast, in their examples applicants have used 3.9 g of diethylamine as an initiator with 7.43 g of L-tyrosine N-carboxyanhydride, 34.61 g of N6-trifluoroacetyl-L-Lysine N-carboxyanhydride, 21.25 g of L-alanine N-carboxyanhydride and 14.83 g of 5-benzyl L-glutamate N-carboxyanhydride in a process conducted at 23° C. to 27° C. for 24 hours to achieve a mixture of polypeptides with a mean molecular weight of 12,700 daltons. The peak molecular weight of the mixture of polypeptides is also affected by the process temperature and reaction time.

In any embodiment of the subject invention, determination of the peak molecular weight of the mixture of polypeptides can be conducted after polymerization of the polypeptide but before removal of either the benzyl protecting group or the trifluoroacetyl protecting group. Alternatively, in any embodiment of the subject invention, the peak molecular weight of the mixture of polypeptides may be determined after removal of the benzyl protecting but before removal of the trifluoroacetyl protecting group. Still another alternative in any embodiment of the subject invention is to determine the peak molecular weight of the mixture of polypeptides after removal of both protecting groups from the polypeptide. Adjustment of the peak molecular weight of the mixture of polypeptides can similarly be performed at the mentioned steps of the process by known techniques such as chromatographic fractionation, filtration, ultrafiltration dialysis, enzymatic hydrolysis or sedimentation.

The subject invention provides a process for making a mixture of acetate salts of polypeptides each of which consisting of glutamic acid, alanine, tyrosine and lysine which provides reduced production of aqueous waste and improved control of the peak molecular weight of the mixture of acetate salts of polypeptides.

1. A process for making a mixture of acetate salts of polypeptides, each of which consisting of glutamic acid, alanine, tyrosine and lysine, wherein the mixture has a desired peak molecular weight, comprising:
a) polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight;
b) removing the benzyl protecting group from the mixture of protected polypeptides by contacting the polypeptides with a hydrogenolysis catalyst and hydrogen to produce a mixture of trifluoroacetyl protected polypeptides, which mixture of polypeptides in unprotected form having the first peak molecular weight;
c) removing the trifluoroacetyl protecting group from the trifluoroacetyl protected polypeptides by contacting the polypeptides with an organic base solution to form a mixture of polypeptides, which mixtures of polypeptides in unprotected form having the first peak molecular weight;
d) removing the free trifluoroacetyl groups and low molecular weight impurities by ultrafiltration to obtain the mixture of polypeptides each of which consisting of glutamic acid, alanine, tyrosine and lysine; and
e) contacting the mixture of polypeptides each of which consisting of glutamic acid, alanine, tyrosine and lysine with an aqueous solution of acetic acid to form the mixture of acetate salts of polypeptides each of which consisting of glutamic acid, alanine, tyrosine and lysine and having the desired peak molecular weight.

2-4. (canceled)

5. The process of claim 1, wherein the desired peak molecular weight is 2,000 daltons to 40,000 daltons.

6. The process of claim 5, wherein the desired peak molecular weight is 4,700 daltons to 11,000 daltons.

7. (canceled)

8. The process of claim 1, wherein the hydrogenolysis catalyst is Palladium/carbon, Raney Nickel, Pt, Pt/C, PtO₂, Pd(OH)₂, Rh/C, or RhCl(PPh₃)₃;
wherein the step of contacting the polypeptides with the hydrogenolysis catalyst is performed in a solvent selected from the group consisting of methanol, ethanol or isopropanol;
wherein the initiator is a primary amine, a dialkyl amine or sodium methoxide;
wherein the amount of initiator is 1% to 10% by weight; and
wherein the organic base in step c) is an aqueous organic base.

9. The process of claim 8, wherein the hydrogenolysis catalyst is Palladium/carbon.

10. The process of claim 9, wherein the weight ratio of protected polypeptide to palladium/carbon catalyst is 10:1.

11. (canceled)

12. The process of claim 9, wherein the solvent is methanol.

13. (canceled)

14. The process of claim 12, wherein the initiator is diethylamine.

15-19. (canceled)

20. The process of claim 14, wherein the aqueous organic base is a primary, secondary or tertiary amine or methanolic ammonia.

21. The process of claim 20, wherein the aqueous organic base is piperidine.

22. A mixture of acetate salts of polypeptides made by the process of claim 1.

23-24. (canceled)

25. In a process for preparing a pharmaceutical composition containing an aqueous mixture of acetate salts of polypeptides each of which consisting of glutamic acid, alanine, tyrosine and lysine, wherein the mixture has a desired peak molecular weight, the improvement comprising making the mixture of acetate salts of polypeptides by the process of claim 1.

26. A process for making a mixture of trifluoroacetyl protected polypeptides, each of which consisting of glutamic acid, alanine, tyrosine and trifluoroacetyllysine, wherein the mixture of polypeptides in unprotected form has a first peak molecular weight, comprising:
a) polymerizing N-carboxyanhydrides of tyrosine, alanine, γ-benzyl glutamate and trifluoroacetyllysine with an initiator in an amount of 0.01% to 20% by weight for a suitable period of time and at a suitable temperature to form a mixture of protected polypeptides, which mixture of polypeptides in unprotected form having a first peak molecular weight; and
b) removing the benzyl protecting group from the mixture of protected polypeptides by contacting the polypeptides with a hydrogenolysis catalyst and hydrogen, to obtain the mixture of trifluoroacetyl protected polypeptides each of which consisting of glutamic acid, alanine, tyrosine and trifluoroacetyllysine and which mixture of polypeptides in unprotected form having the first peak molecular weight.

27. The process of claim 26, wherein the hydrogenolysis catalyst is Palladium/carbon, Raney Nickel, Pt, Pt/C, PtO₂, Pd(OH)₂, Rh/C, or RhCl(PPh₃)₃;
wherein the step of contacting the polypeptides with a hydrogenolysis catalyst is performed in a solvent selected from the group consisting of methanol, ethanol or isopropanol;
wherein the initiator is a primary amine, a dialkyl amine or sodium methoxide; and
wherein the amount of initiator is 1% to 10% by weight.

28. The process of claim 27, wherein the hydrogenolysis catalyst is Palladium/carbon.

29. The process of claim 28, wherein the weight ratio of protected polypeptide to palladium/carbon catalyst is 10:1.

30. (canceled)

31. The process of claim 28, wherein the solvent is methanol.

32. (canceled)

33. The process of claim 31, wherein the initiator is diethylamine.

34-37. (canceled)

38. The process of claim 26, wherein the first peak molecular weight is 2,000 daltons to 40,000 daltons.

39-40. (canceled)

41. A mixture of trifluoroacetyl protected polypeptides each of which consisting of glutamic acid, alanine, tyrosine and trifluoroacetyllysine produced by the process of claim 26.

42. A process of making a mixture of acetate salts of polypeptides, each of which consisting of glutamic acid, alanine, tyrosine and lysine, wherein the mixture has a desired peak molecular weight, comprising:
a) treating the mixture of claim 41 with an organic base solution,
b) removing the free trifluoroacetyl groups and low molecular weight impurities by ultrafiltration to obtain a mixture of polypeptides each of which consisting of glutamic acid, alanine, tyrosine and lysine, and
c) contacting the mixture of polypeptides with an aqueous solution of acetic acid to form the mixture of acetate salts of polypeptides, each of which consisting of glutamic acid, alanine, tyrosine and lysine having the desired peak molecular weight.

43. The process of claim 42, wherein the organic base is an aqueous organic base.

44. The process of claim 43, wherein the aqueous organic base is a primary, secondary or tertiary amine or methanolic ammonia.

45. The process of claim 44, wherein the aqueous organic base is piperidine.
